(19) United States
(12) Patent Application Publication
(10) Pub. No.: US 2023/0360767 A1
(43) Pub. Date: Nov. 9, 2023

(54) Method, System, and Computer Program Product for Estimating Intracranial Pressure Using Near-Infrared Spectroscopy

(71) Applicant: Carnegie Mellon University, Pittsburgh, PA (US)

(72) Inventors: Jana Maria Kainerstofer, Pittsburgh, PA (US); Filip Anders Johan Relander, Pittsburgh, PA (US); Alexander Ruesch, Pittsburgh, PA (US); Matthew A. Smith, Pittsburgh, PA (US)

(21) Appl. No.: 18/131,393

(22) Filed: Apr. 6, 2023

Related U.S. Application Data
(60) Provisional application No. 63/338,069, filed on May 4, 2022.

(51) Int. Cl. G16H 20/40; G06N 20/20

(52) U.S. Cl. CPC G16H 20/40; G06N 20/20

(57) ABSTRACT

The disclosed method includes generating first waveform data using near-infrared spectroscopy (NIRS) to measure at least one light-based signal in a plurality of patients, wherein each waveform of the plurality of waveforms of the first waveform data is associated with at least one blood attribute. The method also includes training a machine learning model based on the first waveform data to produce a trained machine learning model. The method further includes generating second waveform data using NIRS to measure at least one light-based signal in a patient. The method further includes determining an estimated ICP in the patient based on the trained machine learning model. Determining the estimated ICP includes inputting the second waveform data to the trained machine learning model and generating an output from the trained machine learning model including the estimated ICP based on a shape feature of a waveform of the second waveform data.

### CROSS-REFERENCE TO RELATED APPLICATION

This application claims priority to U.S. Provisional Patent Application No. 63/338,069, filed May 4, 2022, the disclosure of which is incorporated by reference herein in its entirety.

### STATEMENT REGARDING FEDERAL FUNDING

This invention was made with government support under Grant No. R21-EB024675 awarded by the National Institutes of Health. The government has certain rights in this invention.

### BACKGROUND

### 1. Technical Field

This disclosure relates generally to neuroscience and, in non-limiting embodiments or aspects, bio-digital methods and systems for monitoring the brain, and uses thereof, including estimating intracranial pressure.

### 2. Technical Considerations

Intracranial pressure (ICP) is the pressure inside the skull. Healthy ICP is maintained through a balance of cerebral blood volume, cerebrospinal fluid (CSF), and brain tissue. Abnormal ICP occurs when an imbalance in one compartment outweighs the compensatory limits of the other two compartments, as explained by the Monro-Kellie doctrine. An increase in ICP can stem from many issues, including brain bleeds, cerebral edema, a mass lesion such as a brain tumor, and others.

The monitoring of ICP may be used to guide the treatment of various diseases and illnesses, such as traumatic brain injury (TBI) and hydrocephalus. TBI, for instance, accounted for over 223,000 hospitalizations in the United States in 2018. Meanwhile, 1 in every 770 babies born in the United States develop congenital hydrocephalus. Accurate ICP monitoring is also pertinent to the estimation of cerebral perfusion pressure (CPP), calculated as the difference between mean arterial pressure (MAP) and ICP. CPP, in turn, may be used in the gauging of cerebral autoregulation (CA), which defines the brain's ability to maintain a near constant blood flow when subject to slow changes in blood pressure, and changes in CPP have been linked to altered neuronal function and neurovascular coupling. ICP measurement may be conducted with an invasive external ventricular drain (EVD). Although the accuracy of an EVD is sensitive to its placement, EVDs have the added benefit of enabling the drainage of excessive CSF.

Invasive ICP monitoring methods, e.g., EVDs, microtransducers, and lumbar puncture (LP) manometries, can be precise in their assessment of ICP in patients, but they are not without risks. EVDs, for example, may include drilling a coronal burr hole and placing a catheter near the third ventricle. This can be difficult in some patients, especially in pediatrics. EVDs and microtransducers may have the potential to cause hemorrhage or infection-related complications. LP manometries measure ICP through CSF pressure in the spinal cord. LP-based ICP sensing may align with EVD-based ICP sensing, but LP-based sensing may rely on extended (e.g., 30 minute) recording periods. In addition, although LPs may be less invasive than EVDs or intraparenchymal probes, LPs carry the risk of infection observed with EVDs and are prone to a higher risk of brain herniation and a larger set of contraindications (compared with EVDs).

Transcranial Doppler (TCD) measurement, a non-invasive technique, measures cerebral blood flow (CBF) velocity, from which peak systolic and diastolic flow rates can be extracted. TCD, however, has been found to lack generalizability. Likewise, the observation of tympanic membrane displacement may coincide with changes in ICP, but the method suffers from poor negative predictive results. Other noninvasive methods using ultrasound, computed tomography (CT), or magnetic resonance imaging (MRI) tend to either be imprecise, fail to work on some patients, only perform binary ICP-level classification, or are not applicable for continuous ICP monitoring applications. CT-based methods, specifically, may not be effective at identifying abnormal CT scans in ICP estimation. Ultrasound methods, meanwhile, are occasionally afflicted by unwanted artifacts and require a standardized technique across patients for reliable ICP sensing.

There is a need in the art for an improved method of accurately estimating intracranial pressure via non-invasive means, which decreases the likelihood of medical complications and difficulty of measuring intracranial pressure.

### SUMMARY

According to some non-limiting embodiments or aspects, provided are a method, system, and computer program product for estimating ICP using near-infrared spectroscopy (NIRS) that overcome one or more deficiencies of the prior art.

According to some non-limiting embodiments or aspects, provided is a computer-implemented method for estimating ICP using NIRS. The computer-implemented method includes generating, with at least one processor, first waveform data using NIRS to measure at least one light-based signal in each patient of a plurality of patients, wherein the first waveform data includes a plurality of waveforms, and wherein each waveform of the plurality of waveforms is associated with at least one blood attribute. The method also includes training, with at least one processor, at least one machine learning model based on the first waveform data to produce at least one trained machine learning model. The at least one trained machine learning model is configured to generate an output of ICP based on one or more waveforms associated with the at least one blood attribute that is input to the at least one trained machine learning model. The method further includes generating, with at least one processor, second waveform data using NIRS to measure at least one light-based signal in a first patient, wherein the second waveform data includes at least one waveform associated with the at least one blood attribute. The method further includes determining, with at least one processor, an estimated ICP in the first patient based on the at least one trained machine learning model. Determining the estimated ICP in the first patient based on the at least one trained machine learning model includes inputting at least a portion of the second waveform data to the at least one trained machine learning model, and generating an output from the at least one trained machine learning model including the estimated ICP based on at least one shape feature of the at least one waveform of the second waveform data.

In some non-limiting embodiments or aspects, generating the first waveform data may include removing, with at least one processor, data outliers from the first waveform data using a preprocessing technique including at least one of the following: normalization, z-score rejection, Kalman filtering, or any combination thereof.

In some non-limiting embodiments or aspects, the method may include comparing, with at least one processor, the estimated ICP to at least one predetermined threshold ICP. The method may further include, in response to the estimated ICP satisfying the at least one predetermined threshold ICP, generating, with at least one processor, at least one alert to a computing device associated with a healthcare personnel providing care to the first patient.

In some non-limiting embodiments or aspects, the method may include performing, with at least one processor, at least one treatment for the first patient based on the estimated ICP.

In some non-limiting embodiments or aspects, the at least one machine learning model may include a random forest model.

In some non-limiting embodiments or aspects, the method may include determining, with at least one processor, MAP data of the first patient. Determining the estimated ICP in the first patient based on the at least one trained machine learning model may further include inputting the MAP data to the at least one trained machine learning model, and generating the output from the at least one trained machine learning model including the estimated ICP based on the MAP data and the at least one shape feature of the at least one waveform of the second waveform data.

In some non-limiting embodiments or aspects, the at least one shape feature of the at least one waveform may include at least one of the following: area under the curve (AUC), x-coordinate of the center of mass (COMₓ), y-coordinate of the center of mass (COM_{y}), peak height, peak width, peak location, or any combination thereof.

In some non-limiting embodiments or aspects, generating the output including the estimated ICP may include generating the output from the at least one trained machine learning model including the estimated ICP based on the at least one shape feature of the at least one waveform of the second waveform data, wherein the at least one shape feature includes a plurality of different shape features.

In some non-limiting embodiments or aspects, generating the first waveform data may further include generating, with at least one processor, a subset of the plurality of waveforms for each patient of the plurality of patients using NIRS to measure a plurality of consecutive cardiac pulses, and determining, with at least one processor, an average cardiac waveform (ACPW) for said each patient based on the subset of the plurality of waveforms.

In some non-limiting embodiments or aspects, the plurality of consecutive cardiac pulses may number in a range of 60 to 120 consecutive cardiac pulses.

In some non-limiting embodiments or aspects, the at least one blood attribute may include at least one of the following: change in oxygenated hemoglobin concentration (ΔHbO), change in total hemoglobin concentration (ΔHbT), or any combination thereof.

According to some non-limiting embodiments or aspects, provided is a system for estimating ICP using NIRS. The system includes at least one processor, which is programmed or configured to generate first waveform data using NIRS to measure at least one light-based signal in each patient of a plurality of patients, wherein the first waveform data includes a plurality of waveforms, and wherein each waveform of the plurality of waveforms is associated with at least one blood attribute. The at least one processor is also programmed or configured to train at least one machine learning model based on the first waveform data to produce at least one trained machine learning model. The at least one trained machine learning model is configured to generate an output of ICP based on one or more waveforms associated with the at least one blood attribute that is input to the at least one trained machine learning model. The at least one processor is further programmed or configured to generate second waveform data using NIRS to measure at least one light-based signal in a first patient, wherein the second waveform data includes at least one waveform associated with the at least one blood attribute. The at least one processor is further programmed or configured to determine an estimated ICP in the first patient based on the at least one trained machine learning model. While determining the estimated ICP in the first patient based on the at least one trained machine learning model, the at least one processor is further programmed or configured to input at least a portion of the second waveform data to the at least one trained machine learning model, and generate an output from the at least one trained machine learning model including the estimated ICP based on at least one shape feature of the at least one waveform of the second waveform data.

In some non-limiting embodiments or aspects, while generating the first waveform data, the at least one processor may be programmed or configured to remove data outliers from the first waveform data using a preprocessing technique including at least one of the following: normalization, z-score rejection, Kalman filtering, or any combination thereof.

In some non-limiting embodiments or aspects, the at least one processor may be further programmed or configured to compare the estimated ICP to at least one predetermined threshold ICP and, in response to the estimated ICP satisfying the at least one predetermined threshold ICP, generate at least one alert to a computing device associated with a healthcare personnel providing care to the first patient.

In some non-limiting embodiments or aspects, the at least one processor may be further programmed or configured to perform at least one treatment for the first patient based on the estimated ICP.

In some non-limiting embodiments or aspects, the at least one machine learning model may include a random forest model.

In some non-limiting embodiments or aspects, the at least one processor may be further programmed or configured to determine mean arterial pressure (MAP) data of the first patient. While determining the estimated ICP in the first patient based on the at least one trained machine learning model, the at least one processor may be programmed or configured to input the MAP data to the at least one trained machine learning model, and generate the output from the at least one trained machine learning model including the estimated ICP based on the MAP data and the at least one shape feature of the at least one waveform of the second waveform data.

In some non-limiting embodiments or aspects, the at least one shape feature of the at least one waveform may include at least one of the following: area under the curve (AUC), x-coordinate of the center of mass (COMₓ), y-coordinate of the center of mass (COM_{y}), peak height, peak width, peak location, or any combination thereof.

In some non-limiting embodiments or aspects, while generating the output including the estimated ICP, the at least one processor may be programmed or configured to generate the output from the at least one trained machine learning model including the estimated ICP based on the at least one shape feature of the at least one waveform of the second waveform data, wherein the at least one shape feature includes a plurality of different shape features.

In some non-limiting embodiments or aspects, while generating the first waveform data, the at least one processor may be programmed or configured to generate a subset of the plurality of waveforms for each patient of the plurality of patients using NIRS to measure a plurality of consecutive cardiac pulses, and determine an ACPW for said each patient based on the subset of the plurality of waveforms.

In some non-limiting embodiments or aspects, the plurality of consecutive cardiac pulses may number in a range of 60 to 120 consecutive cardiac pulses.

In some non-limiting embodiments or aspects, the at least one blood attribute may include at least one of the following: change in oxygenated hemoglobin concentration (ΔHbO), change in total hemoglobin concentration (ΔHbT), or any combination thereof.

According to some non-limiting embodiments or aspects, provided is a computer program product for estimating ICP using NIRS. The computer program product includes at least one non-transitory computer-readable medium including one or more instructions that, when executed by at least one processor, cause the at least one processor to generate first waveform data NIRS to measure at least one light-based signal in each patient of a plurality of patients, wherein the first waveform data includes a plurality of waveforms, and wherein each waveform of the plurality of waveforms is associated with at least one blood attribute. The one or more instructions further cause the at least one processor to train at least one machine learning model based on the first waveform data to produce at least one trained machine learning model. The at least one trained machine learning model is configured to generate an output of ICP based on one or more waveforms associated with the at least one blood attribute that is input to the at least one trained machine learning model. The one or more instructions further cause the at least one processor to generate second waveform data using NIRS to measure at least one light-based signal in a first patient, wherein the second waveform data includes at least one waveform associated with the at least one blood attribute. The one or more instructions further cause the at least one processor to determine an estimated ICP in the first patient based on the at least one trained machine learning model. The one or more instructions that cause the at least one processor to determine the estimated ICP in the first patient based on the at least one trained machine learning model cause the at least one processor to input at least a portion of the second waveform data to the at least one trained machine learning model, and generate an output from the at least one trained machine learning model including the estimated ICP based on at least one shape feature of the at least one waveform of the second waveform data.

In some non-limiting embodiments or aspects, the one or more instructions that cause the at least one processor to generate the first waveform data may cause the at least one processor to remove data outliers from the first waveform data using a preprocessing technique including at least one of the following: normalization, z-score rejection, Kalman filtering, or any combination thereof.

In some non-limiting embodiments or aspects, the one or more instructions may further cause the at least one processor to compare the estimated ICP to at least one predetermined threshold ICP. The one or more instructions may further cause that least one processor to, in response to the estimated ICP satisfying the at least one predetermined threshold ICP, generate at least one alert to a computing device associated with a healthcare personnel providing care to the first patient.

In some non-limiting embodiments or aspects, the one or more instructions may further cause the at least one processor to perform at least one treatment for the first patient based on the estimated ICP.

In some non-limiting embodiments or aspects, the at least one machine learning model may include a random forest model.

In some non-limiting embodiments or aspects, the one or more instructions may further cause the at least one processor to determine MAP data of the first patient. The one or more instructions that cause the at least one processor to determine the estimated ICP in the first patient based on the at least one trained machine learning model may cause the at least one processor to input the MAP data to the at least one trained machine learning model, and generate the output from the at least one trained machine learning model including the estimated ICP based on the MAP data and the at least one shape feature of the at least one waveform of the second waveform data.

In some non-limiting embodiments or aspects, the at least one shape feature of the at least one waveform may include at least one of the following: area under the curve (AUC), x-coordinate of the center of mass (COMₓ), y-coordinate of the center of mass (COM_{y}), peak height, peak width, peak location, or any combination thereof.

In some non-limiting embodiments or aspects, the one or more instructions that cause the at least one processor to generate the output including the estimated ICP may cause the at least one processor to generate the output from the at least one trained machine learning model including the estimated ICP based on the at least one shape feature of the at least one waveform of the second waveform data, wherein the at least one shape feature includes a plurality of different shape features.

In some non-limiting embodiments or aspects, the one or more instructions that cause the at least one processor to generate the first waveform data may cause the at least one processor to generate a subset of the plurality of waveforms for each patient of the plurality of patients using NIRS to measure a plurality of consecutive cardiac pulses, and determine an ACPW for said each patient based on the subset of the plurality of waveforms.

In some non-limiting embodiments or aspects, the plurality of consecutive cardiac pulses may number in a range of 60 to 120 consecutive cardiac pulses.

In some non-limiting embodiments or aspects, the at least one blood attribute may include at least one of the following: change in oxygenated hemoglobin concentration (ΔHbO), change in total hemoglobin concentration (ΔHbT), or any combination thereof.

Other non-limiting embodiments or aspects of the present disclosure will be set forth in the following numbered clauses:
Clause 1: A computer-implemented method comprising: generating, with at least one processor, first waveform data using near-infrared spectroscopy (NIRS) to measure at least one light-based signal in each patient of a plurality of patients, wherein the first waveform data comprises a plurality of waveforms, and wherein each waveform of the plurality of waveforms is associated with at least one blood attribute; training, with at least one processor, at least one machine learning model based on the first waveform data to produce at least one trained machine learning model, wherein the at least one trained machine learning model is configured to generate an output of intracranial pressure (ICP) based on one or more waveforms associated with the at least one blood attribute that is input to the at least one trained machine learning model; generating, with at least one processor, second waveform data using NIRS to measure at least one light-based signal in a first patient, wherein the second waveform data comprises at least one waveform associated with the at least one blood attribute; and determining, with at least one processor, an estimated ICP in the first patient based on the at least one trained machine learning model, wherein determining the estimated ICP in the first patient based on the at least one trained machine learning model comprises: inputting at least a portion of the second waveform data to the at least one trained machine learning model; and generating an output from the at least one trained machine learning model comprising the estimated ICP based on at least one shape feature of the at least one waveform of the second waveform data.
Clause 2: The method of clause 1, wherein generating the first waveform data further comprises: removing, with at least one processor, data outliers from the first waveform data using a preprocessing technique comprising at least one of the following: normalization, z-score rejection, Kalman filtering, or any combination thereof.
Clause 3: The method of clause 1 or clause 2, further comprising: comparing, with at least one processor, the estimated ICP to at least one predetermined threshold ICP; and in response to the estimated ICP satisfying the at least one predetermined threshold ICP, generating, with at least one processor, at least one alert to a computing device associated with a healthcare personnel providing care to the first patient.
Clause 4: The method of any one of clauses 1-3, further comprising performing, with at least one processor, at least one treatment for the first patient based on the estimated ICP.
Clause 5: The method of any one of clauses 1-4, wherein the at least one machine learning model comprises a random forest model.
Clause 6: The method of any one of clauses 1-5, further comprising determining, with at least one processor, mean arterial pressure (MAP) data of the first patient, wherein determining the estimated ICP in the first patient based on the at least one trained machine learning model further comprises: inputting the MAP data to the at least one trained machine learning model; and generating the output from the at least one trained machine learning model comprising the estimated ICP based on the MAP data and the at least one shape feature of the at least one waveform of the second waveform data.
Clause 7: The method of any one of clauses 1-6, wherein the at least one shape feature of the at least one waveform comprises at least one of the following: area under the curve (AUC), x-coordinate of the center of mass (COMₓ), y-coordinate of the center of mass (COM_{y}), peak height, peak width, peak location, or any combination thereof.
Clause 8: The method of any one of clauses 1-7, wherein generating the output comprising the estimated ICP further comprises: generating the output from the at least one trained machine learning model comprising the estimated ICP based on the at least one shape feature of the at least one waveform of the second waveform data, wherein the at least one shape feature comprises a plurality of different shape features.
Clause 9: The method of any one of clauses 1-8, wherein generating the first waveform data further comprises: generating, with at least one processor, a subset of the plurality of waveforms for each patient of the plurality of patients using NIRS to measure a plurality of consecutive cardiac pulses; and determining, with at least one processor, an average cardiac waveform (ACPW) for said each patient based on the subset of the plurality of waveforms.
Clause 10: The method of clause 9, wherein the plurality of consecutive cardiac pulses numbers in a range of 60 to 120 consecutive cardiac pulses.
Clause 11: The method of any one of clauses 1-10, wherein the at least one blood attribute comprises at least one of the following: change in oxygenated hemoglobin concentration (ΔHbO), change in total hemoglobin concentration (ΔHbT), or any combination thereof.
Clause 12: A system comprising at least one processor programmed or configured to: generate first waveform data using near-infrared spectroscopy (NIRS) to measure at least one light-based signal in each patient of a plurality of patients, wherein the first waveform data comprises a plurality of waveforms, and wherein each waveform of the plurality of waveforms is associated with at least one blood attribute; train at least one machine learning model based on the first waveform data to produce at least one trained machine learning model, wherein the at least one trained machine learning model is configured to generate an output of intracranial pressure (ICP) based on one or more waveforms associated with the at least one blood attribute that is input to the at least one trained machine learning model; generate second waveform data using NIRS to measure at least one light-based signal in a first patient, wherein the second waveform data comprises at least one waveform associated with the at least one blood attribute; and determine an estimated ICP in the first patient based on the at least one trained machine learning model, wherein, while determining the estimated ICP in the first patient based on the at least one trained machine learning model, the at least one processor is further programmed or configured to: input at least a portion of the second waveform data to the at least one trained machine learning model; and generate an output from the at least one trained machine learning model comprising the estimated ICP based on at least one shape feature of the at least one waveform of the second waveform data.
Clause 13: The system of clause 12, wherein, while generating the first waveform data, the at least one processor is programmed or configured to: remove data outliers from the first waveform data using a preprocessing technique comprising at least one of the following: normalization, z-score rejection, Kalman filtering, or any combination thereof.
Clause 14: The system of clause 12 or clause 13, wherein the at least one processor is further programmed or configured to: compare the estimated ICP to at least one predetermined threshold ICP; and, in response to the estimated ICP satisfying the at least one predetermined threshold ICP, generate at least one alert to a computing device associated with a healthcare personnel providing care to the first patient.
Clause 15: The system of any one of clauses 12-14, wherein the at least one processor is further programmed or configured to perform at least one treatment for the first patient based on the estimated ICP.
Clause 16: The system of any one of clauses 12-15, wherein the at least one machine learning model comprises a random forest model.
Clause 17: The system of any one of clauses 12-16, wherein the at least one processor is further programmed or configured to determine mean arterial pressure (MAP) data of the first patient, and wherein, while determining the estimated ICP in the first patient based on the at least one trained machine learning model, the at least one processor is programmed or configured to: input the MAP data to the at least one trained machine learning model; and generate the output from the at least one trained machine learning model comprising the estimated ICP based on the MAP data and the at least one shape feature of the at least one waveform of the second waveform data.
Clause 18: The system of any one of clauses 12-17, wherein the at least one shape feature of the at least one waveform comprises at least one of the following: area under the curve (AUC), x-coordinate of the center of mass (COMₓ), y-coordinate of the center of mass (COM_{y}), peak height, peak width, peak location, or any combination thereof.
Clause 19: The system of any one of clauses 12-18, wherein, while generating the output comprising the estimated ICP, the at least one processor is programmed or configured to: generate the output from the at least one trained machine learning model comprising the estimated ICP based on the at least one shape feature of the at least one waveform of the second waveform data, wherein the at least one shape feature comprises a plurality of different shape features.
Clause 20: The system of any one of clauses 12-19, wherein, while generating the first waveform data, the at least one processor is programmed or configured to: generate a subset of the plurality of waveforms for each patient of the plurality of patients using NIRS to measure a plurality of consecutive cardiac pulses; and determine an average cardiac waveform (ACPW) for said each patient based on the subset of the plurality of waveforms.
Clause 21: The system of clause 20, wherein the plurality of consecutive cardiac pulses numbers in a range of 60 to 120 consecutive cardiac pulses.
Clause 22: The system of any one of clauses 12-21, wherein the at least one blood attribute comprises at least one of the following: change in oxygenated hemoglobin concentration (ΔHbO), change in total hemoglobin concentration (ΔHbT), or any combination thereof.
Clause 23: A computer program product comprising at least one non-transitory computer-readable medium comprising one or more instructions that, when executed by at least one processor, cause the at least one processor to: generate first waveform data using near-infrared spectroscopy (NIRS) to measure at least one light-based signal in each patient of a plurality of patients, wherein the first waveform data comprises a plurality of waveforms, and wherein each waveform of the plurality of waveforms is associated with at least one blood attribute; train at least one machine learning model based on the first waveform data to produce at least one trained machine learning model, wherein the at least one trained machine learning model is configured to generate an output of intracranial pressure (ICP) based on one or more waveforms associated with the at least one blood attribute that is input to the at least one trained machine learning model; generate second waveform data using NIRS to measure at least one light-based signal in a first patient, wherein the second waveform data comprises at least one waveform associated with the at least one blood attribute; and determine an estimated ICP in the first patient based on the at least one trained machine learning model, wherein the one or more instructions that cause the at least one processor to determine the estimated ICP in the first patient based on the at least one trained machine learning model cause the at least one processor to: input at least a portion of the second waveform data to the at least one trained machine learning model; and generate an output from the at least one trained machine learning model comprising the estimated ICP based on at least one shape feature of the at least one waveform of the second waveform data.
Clause 24: The computer program product of clause 23, wherein the one or more instructions that cause the at least one processor to generate the first waveform data cause the at least one processor to: remove data outliers from the first waveform data using a preprocessing technique comprising at least one of the following: normalization, z-score rejection, Kalman filtering, or any combination thereof.
Clause 25: The computer program product of clause 23 or clause 24, wherein the one or more instructions further cause the at least one processor to: compare the estimated ICP to at least one predetermined threshold ICP; and, in response to the estimated ICP satisfying the at least one predetermined threshold ICP, generate at least one alert to a computing device associated with a healthcare personnel providing care to the first patient.
Clause 26: The computer program product of any one of clauses 23-25, wherein the one or more instructions further cause the at least one processor to perform at least one treatment for the first patient based on the estimated ICP.
Clause 27: The computer program product of any one of clauses 23-26, wherein the at least one machine learning model comprises a random forest model.
Clause 28: The computer program product of any one of clauses 23-27, wherein the one or more instructions further cause the at least one processor to determine mean arterial pressure (MAP) data of the first patient, and wherein the one or more instructions that cause the at least one processor to determine the estimated ICP in the first patient based on the at least one trained machine learning model cause the at least one processor to: input the MAP data to the at least one trained machine learning model; and generate the output from the at least one trained machine learning model comprising the estimated ICP based on the MAP data and the at least one shape feature of the at least one waveform of the second waveform data.
Clause 29: The computer program product of any one of clauses 23-28, wherein the at least one shape feature of the at least one waveform comprises at least one of the following: area under the curve (AUC), x-coordinate of the center of mass (COMₓ), y-coordinate of the center of mass (COM_{y}), peak height, peak width, peak location, or any combination thereof.
Clause 30: The computer program product of any one of clauses 23-29, wherein the one or more instructions that cause the at least one processor to generate the output comprising the estimated ICP cause the at least one processor to: generate the output from the at least one trained machine learning model comprising the estimated ICP based on the at least one shape feature of the at least one waveform of the second waveform data, wherein the at least one shape feature comprises a plurality of different shape features.
Clause 31: The computer program product of any one of clauses 23-30, wherein the one or more instructions that cause the at least one processor to generate the first waveform data cause the at least one processor to: generate a subset of the plurality of waveforms for each patient of the plurality of patients using NIRS to measure a plurality of consecutive cardiac pulses; and determine an average cardiac waveform (ACPW) for said each patient based on the subset of the plurality of waveforms.
Clause 32: The computer program product of clause 31, wherein the plurality of consecutive cardiac pulses numbers in a range of 60 to 120 consecutive cardiac pulses.
Clause 33: The computer program product of any one of clauses 23-32, wherein the at least one blood attribute comprises at least one of the following: change in oxygenated hemoglobin concentration (ΔHbO), change in total hemoglobin concentration (ΔHbT), or any combination thereof.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present disclosure. As used in the specification and the claims, the singular form of “a,” “an,” and “the” include plural referents unless the context clearly dictates otherwise.

### BRIEF DESCRIPTION OF THE DRAWINGS

The patent or application file contains at least one drawing executed in color. Copies of this patent or patent application publication with color drawings will be provided by the Office upon request and payment of the necessary fee.

Additional advantages and details of the disclosure are explained in greater detail below with reference to the exemplary embodiments or aspects that are illustrated in the accompanying schematic figures, in which:

FIG. 1 is an illustrative diagram of a setup for generating waveform data from a patient, according to some non-limiting embodiments or aspects of the present disclosure;

FIG. 2 depicts waveform graphs produced from light-based signals measured using NIRS and corresponding to certain blood attributes, according to some non-limiting embodiments or aspects of the present disclosure;

FIG. 3A depicts exemplary shape features of waveforms, according to some non-limiting embodiments or aspects of methods for estimating ICP using NIRS;

FIG. 3B depicts exemplary shape features of waveforms, according to some non-limiting embodiments or aspects of methods for estimating ICP using NIRS;

FIG. 4A depicts a histogram of ICP distribution for training and test datasets for light-based signals associated with two blood attributes, according to some non-limiting embodiments or aspects of the present disclosure;

FIG. 4B depicts a correlation plot illustrating correlation between estimated ICP (determined from light-based signals associated with a first blood attribute, ΔHbO) and invasively determined ICP, according to some non-limiting embodiments or aspects of the present disclosure;

FIG. 4C depicts a correlation plot illustrating correlation between estimated ICP (determined from light-based signals associated with a second blood attribute, ΔHbT) and invasively determined ICP, according to some non-limiting embodiments or aspects of the present disclosure;

FIG. 5A depicts a Bland-Altman plot for ΔHbO with a histogram illustrating the distribution of data points across the Bland-Altman plot, according to some non-limiting embodiments or aspects of the present disclosure;

FIG. 5B depicts a Bland-Altman plot for ΔHbT with a histogram illustrating the distribution of data points across the Bland-Altman plot, according to some non-limiting embodiments or aspects of the present disclosure;

FIG. 6 depicts histograms of bootstrapped r² scores for ΔHbO, ΔHbT, and CBF demonstrating performance of those blood attributes as modalities for use in training a machine learning model to estimate ICP, according to some non-limiting embodiments or aspects of the present disclosure;

FIG. 7A depicts histograms of feature statistical importance in estimating ICP based on waveforms associated with the blood attribute of ΔHbO, according to some non-limiting embodiments or aspects of the present disclosure;

FIG. 7B depicts histograms of feature statistical importance in estimating ICP based on waveforms associated with the blood attribute of ΔHbT, according to some non-limiting embodiments or aspects of the present disclosure;

FIG. 8 is a diagram of a non-limiting embodiment or aspect of an environment in which systems, devices, products, apparatus, and/or methods, described herein, may be implemented, according to the principles of the present disclosure;

FIG. 9 is a diagram of one or more components, devices, and/or systems, according to some non-limiting embodiments or aspects of the present disclosure; and

FIG. 10 is a flowchart of a method for estimating ICP using NIRS, according to some non-limiting embodiments or aspects of the present disclosure.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the disclosure as it is oriented in the drawing figures. However, it is to be understood that the disclosure may assume various alternative variations and step sequences, except where expressly specified to the contrary. It is also to be understood that the specific devices and processes illustrated in the attached drawings, and described in the following specification, are simply exemplary embodiments or aspects of the disclosure. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. The phase “based on” may also mean “in response to” where appropriate.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, and/or the like.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, “interface” refers, in the context of programming and software modules, to the languages, codes and messages that programs or modules use to communicate with each other and to the hardware, and includes computer code or other data stored on a computer-readable medium that may be executed by a processor to facilitate the interaction between software modules. In some aspects of the methods and systems described herein, software modules, such as the variant calling module, the tumor phylogeny or modules and the machine learning modules are designed as separate software components, modules, or engines, with each requiring specific data input formats, and providing specific data output formats, and, in non-limiting examples, an interface may be used to facilitate such communication between components.

As used herein, the term “graphical user interface” or “GUI” refers to a generated display with which a user may interact, either directly or indirectly (e.g., through a keyboard, mouse, touchscreen, and/or the like).

As used herein, the term “server” may refer to or include one or more computing devices that are operated by or facilitate communication and processing for multiple parties in a network environment, such as the internet, although it will be appreciated that communication may be facilitated over one or more public or private network environments and that various other arrangements are possible. Further, one or more computing devices (e.g., servers, mobile devices, etc.) directly or indirectly communicating in the network environment may constitute a “system.” Reference to “a server” or “a processor,” as used herein, may refer to a previously recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates and grammatical variants of those words or phrases.

As used herein, the terms “comprising,” “comprise” or “comprised,” and variations thereof, in reference to elements of an item, composition, apparatus, method, process, system, claim etc. are intended to be open-ended, meaning that the item, composition, apparatus, method, process, system, claim etc. includes those elements and other elements can be included and still fall within the scope/definition of the described item, composition, apparatus, method, process, system, claim, etc.

As used herein, the terms “patient” or “subject” refer to members of the animal kingdom, including, but not limited to, human beings.

The methods, systems, and computer program products described herein provide numerous technical advantages in systems for estimating ICP. First, the described techniques relate to estimating ICP using NIRS, which is a non-invasive measurement technique and does not require invasive measurement of ICP. As such, the described techniques have lower risk of infection and complications from measurement. Furthermore, the described techniques relate to generating waveform data based on NIRS-measured light-based signals that are associated with (e.g., converted to) blood attributes like ΔHbO and ΔHbT, which have been shown to have good performance in training a model to predict ICP from such non-invasive NIRS measurement. Using waveform data generated from NIRS measurement also reduces overall time for data capture and estimation, given that the NIRS measurement is non-invasive and does not require invasive surgical setup. Additionally, the described techniques related to using shape features of waveforms as engineering features for trained machine learning models, by which ICP may be estimated from patient waveform data, have demonstrated good performance in estimate accuracy relative to known techniques, but have the advantage of being overall cheaper and simpler to implement than such known techniques (given a basis in NIRS). Finally, random forest regressor models also have low computational cost for use as the trained machine learning models, producing effective estimates of ICP while also preventing overfitting of data.

In some non-limiting embodiments or aspects, hemodynamic-based methods that use diffuse optical devices, such as near-infrared spectroscopy (NIRS), may be useful for noninvasive ICP monitoring. ICP may be estimated from cardiac waveform features of light-based signals generated with NIRS. Relative changes in oxyhemoglobin concentrations correlate with relative changes in ICP. NIRS-based ICP estimation is favorable to other noninvasive ICP monitoring methods, including TCD, CT, MRI, ultrasound, and diffuse correlation spectroscopy (DCS), due to its improved efficiency, improved accuracy, lower cost, user independence, and bedside compatibility for long-term monitoring.

For the purposes of evaluating the disclosed techniques, oxyhemoglobin and deoxyhemoglobin concentration changes were recorded at various ICP levels with NIRS across eight nonhuman primates (NHPs). Cardiac pulse waveforms were extracted and processed. Their features were used to train a machine learning algorithm for noninvasive ICP estimation. Eight anesthetized NHPs (NHPs 1 to 4 Macaca mulatta aged 7.9±1.5 years, weighing 9.4±0.7 kg, and NHPs 5 to 8 Macaca fascicularis aged 4.2±0.9 years, weighing 5.1±2.1 kg) were used for the experiments. Each NHP was sedated throughout experimentation. Prior to their transport into the experimentation room, the NHPs were sedated with 20-mg/kg ketamine. In some animals, an additional 0.04-mg/kg of atropine and 1-mg/kg diazepam were administered. During the experiment, the monkeys were anesthetized with a combination of 0.6% to 1.5% isoflurane (ISO) and 10- to 25.6-μg/kg/h fentanyl. Additionally, 0.1-mg/kg/h of vecuronium bromide paralytic was administered intravenously. NHPs were ventilated at 0.18 to 0.4 Hz. The ventilation frequency was kept constant for the duration of the experiment for each NHP.

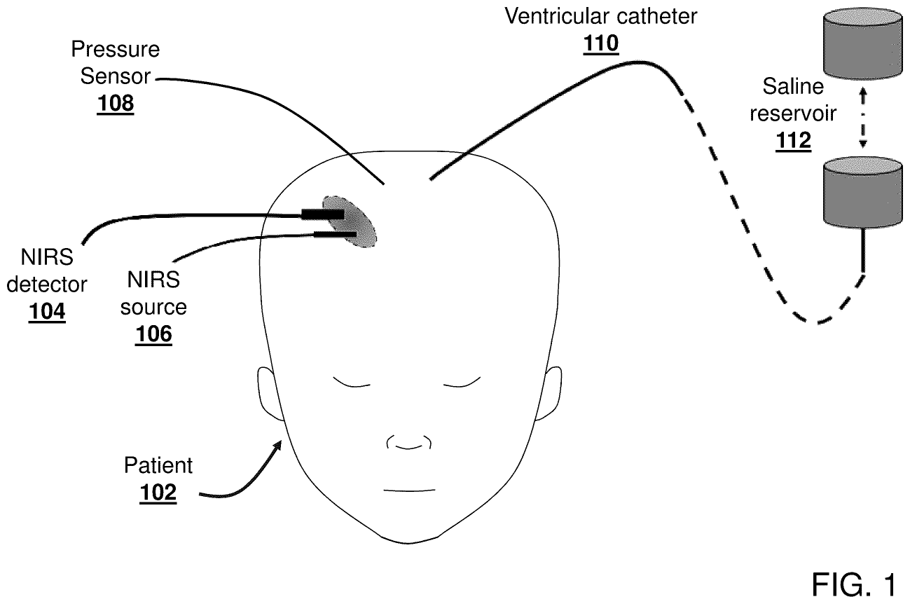

Referring to FIG. 1, depicted is an illustrative diagram of a setup for generating waveform data from a patient 102 using NIRS, according to some non-limiting embodiments or aspects of the present disclosure. Depicted is NIRS placement on the skull above the prefrontal cortex of patient 102. The NIRS setup shows a defined distance between NIRS detector 104 and NIRS source 106. For validation, ICP was measured invasively using a parenchymal pressure sensor 108. Saline was administered from saline reservoir 112 using ventricular catheter 110. ICP was regulated by adjusting the height of saline reservoir 112. A frequency-domain NIRS system (e.g., OxiplexTS, ISS Inc., United States) operating at 690 nm and 830 nm, was used to measure cerebral hemoglobin concentration changes. The differential path length factor (DPF) ratio was held constant between all animals at DPF₆₉₀/DPF₈₃₀=1.1. Although DPF differences are likely between animals, the cardiac waveform shape is not expected to be influenced significantly. To overcome magnitude differences between animals, signals were normalized. Optical probes were placed directly on the skull of the animals, above the right prefrontal cortex. For NHPs 1 to 3, the NIRS source-detector distance was 2.2 cm, and for NHPs 4 to 8, the NIRS source-detector distance was 1.5 cm.

With further reference to FIG. 1, the data for this evaluation were based on retrospective analysis. Differences in source detector distances were due to variations in overall test designs between animals. Light intensity changes of the light-based signals measured by NIRS were recorded at a sampling frequency of 50 Hz and converted to changes in oxygenated hemoglobin concentration (ΔHbO), deoxygenated hemoglobin concentration (ΔHb), and total hemoglobin concentration (ΔHbT=ΔHbO+ΔHb) using the modified Beer-Lambert's law.

Arterial blood pressure (ABP) was recorded with an MPR1 Datalogger (Raumedic Helmbrechts, Germany) using an arterial line placed in the carotid artery. ICP was altered using a catheter (Lumbar catheter, Medtronic, Minneapolis, Minnesota, United States) placed in the lateral ventricle of the brain, with the other end connected to a saline reservoir 112, as shown in FIG. 1. A change in the height of the saline reservoir 112 resulted in a pressure change in the head (e.g., simulating hydrocephalus). ICP was measured using a parenchymal pressure sensor 108 recorded by the MPR1 Datalogger (Raumedic Helmbrechts, Germany). Both ICP and ABP were recorded at 100 Hz.

Each NHP experiment, lasting about 22.6 hours (with a standard deviation of 2.3 hours), was split between 7 and 10 separate trials. Each trial was about 90 minutes long and corresponded to a particular ICP level, elevated using saline infusion. Induced ICP ranged between 5 and 60 mmHg, with natural ICP fluctuation during experimentation deviating slightly beyond these limits. The distribution of both induced and naturally fluctuating ICP levels was observed to be predominantly between 5 and 30 mmHg. Before ICP elevation, a recording at baseline ICP was also conducted.

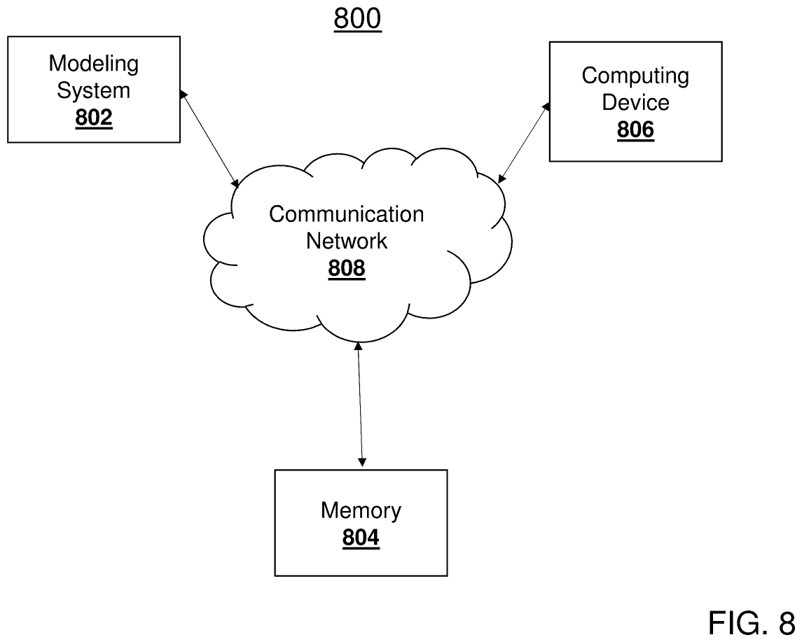

In some non-limiting embodiments or aspects, for implementation purposes, a similar setup as shown in FIG. 1 may be used for gathering waveform data for use in training machine learning models and using trained machine-learning models. Implementation may include NIRS detector 104 and NIRS source 106, placed on the skull of patient 102. NIRS detector 104 and NIRS source 106 may be a part of a NIRS system, which may be associated with or included in a modeling system 802, as illustrated in FIG. 8.

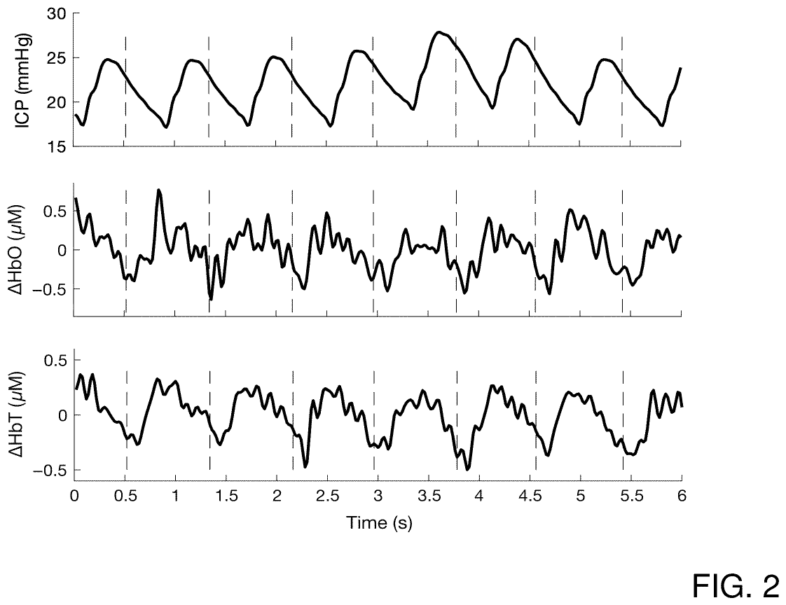

Referring to FIG. 2, FIG. 2 depicts waveform graphs produced from using NIRS to measure light-based signals associated with certain blood attributes in patients, according to non-limiting embodiments or aspects of the present disclosure. In particular, FIG. 2 depicts filtered time trace examples of ICP, ΔHbO, and ΔHbT signals. Dashed lines indicate QRS complex peaks (e.g., which may designate the intervals of consecutive cardiac pulses). As shown in FIG. 2, the waveform graphs illustrates examples of 50 Hz ΔHbO, ΔHbT, and ICP signals. In analysis, a total of 19,000 ΔHbO and 19,258 ΔHbT average cardiac waveforms (ACPWs) were sampled from 8 NHPs for feature extraction. All ACPWs represented an ICP range of 0 to 30 mmHg.

To achieve data alignment, analog markers in the form of voltage spikes were sent to the auxiliary ports of the NIRS and MPR1 Datalogger devices. In NHPs 1 to 3, an amplifier circuit was set to register a cardiac pulse when the electrocardiogram (EKG) signal exceeded an empirically determined threshold that defined the R peak of the QRS complex. When an R peak was detected, a synchronization pulse was sent to the MPR1 Datalogger. This signal was sampled at 100 Hz. For NHPs 4 to 8, EKG was measured through a separate device at 1000 Hz. A maximal overlap discrete wavelet transform was run across the EKG signal to enhance the QRS complex features of the signal (the QRS complex represents ventricular polarization). MATLAB's (MATLAB R2020b, The MathWorks Inc., Natick, Massachusetts, United States) “findpeaks” function was used to index the time point of the peak of each enhanced QRS complex. The respective indices represent the R peak of the QRS complex. During data collection, occasional laser instabilities were observed. Time points in the NIRS-based signals, for which instabilities were visually observed, were removed. All signals not originally sampled at 50 Hz (e.g., ABP, ICP, and QRS complex peak indices) were downsampled to 50 Hz to match the sampling frequency of the NIRS signal. It will be appreciated that similar techniques may be executed, in an implementation environment, to generate waveform data using NIRS for training machine learning models based on one or more blood attributes.

Pre-processing and aggregation may be useful for reducing noise in signals for generating waveform data. To reduce noise in the NIRS-measured light-based signals, 120 consecutive cardiac pulses were averaged, and an ACPW was extracted. Because the heart rate of the animals varied, 120 pulses corresponded to between 39 and 78 seconds across all animals. The ICP values during the 120 pulses were also averaged. The 120-pulse averaging window was moved 20 pulses at a time (resulting in an 83.3% window-to-window overlap) along the entire signal of each trial. All ACPWs were normalized in time and amplitude. With the help of spline interpolation, the length of each ACPW was normalized to 66 data points, corresponding to 1.32 seconds. ACPW length was measured between two consecutive diastoles. The height of each ACPW (representing the amplitude of ΔHbO and ΔHbT) was normalized to between 0 and 1. Normalization of the x- and y-axis removed ACPW length as a feature and, thus, removed heart rate as a feature of the waveform. Heart rate was removed as a feature because heart rate changes may be independent of ICP. To remove outliers, a z-score rejection method was applied to all ACPWs. For each trial, a z-score was calculated across all averaged pulses in the trial. This averaged z-score was then compared against each ACPW. Any ΔHbO or ΔHbT ACPW with a z-score greater than 3 was rejected. It will be appreciated that the number of consecutive cardiac pulses to average for each ACPW may be in a range selected for its ability to produce goods results while not excessively requiring computer resources and time to collect data for averaging (e.g., 60 to 120 consecutive cardiac pulses, 60 to 100 consecutive cardiac pulses, 100 to 120 consecutive cardiac pulses, 120 to 150 consecutive cardiac pulses, etc.).

A Kalman filter was then used to further improve signal quality. The adaptive filter was applied to the ACPWs of each signal for each trial. Each ACPW was compared with the ideal trial pulse produced by the Kalman filter. ACPWs were corrected based on their error from the ideal pulse. In the correction method, the Kalman filter's parameters defined the weight given to the Kalman-produced ideal pulse and the weight given to the calculated ACPW-to-ideal-pulse error. These parameters were set empirically. The output of the Kalman filter procedure was a set of high signal-to-noise ratio (SNR) ACPWs with feature morphology reflecting changes in ICP. ACPWs reflecting ICP values above 30 mmHg were removed due to their scarcity. It will be appreciated that the above techniques may be useful, in an implementation environment, to filter and denoise waveform data for use in training machine learning models.

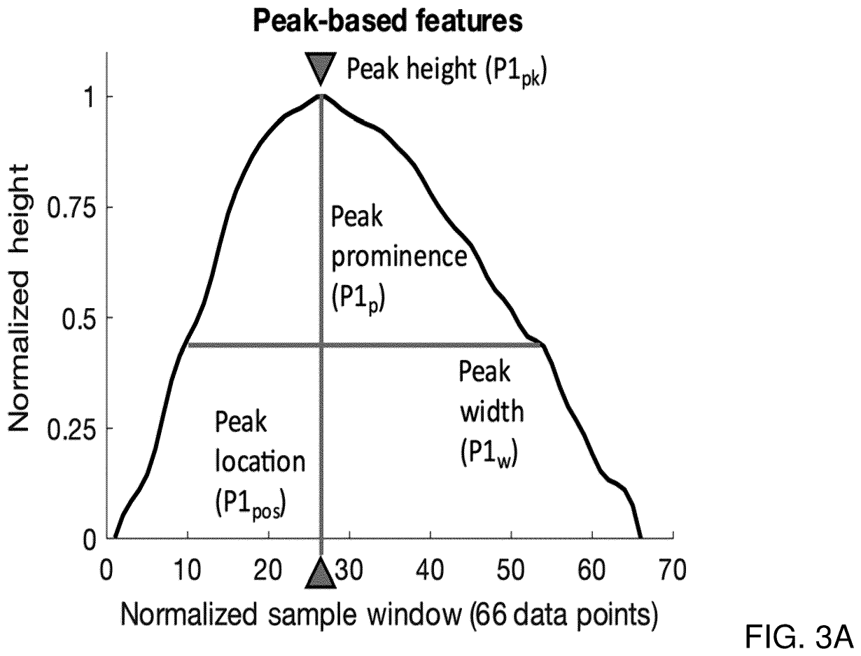

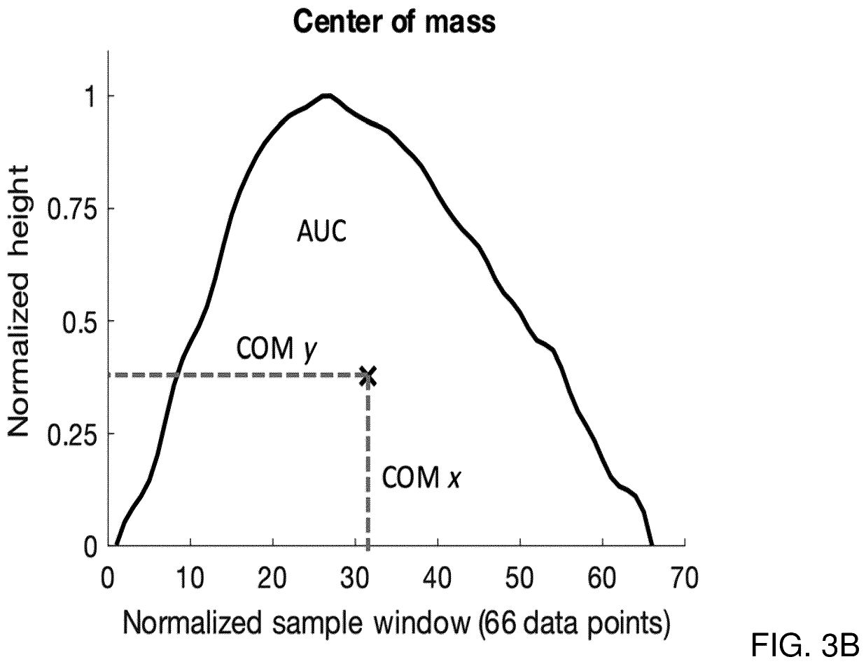

Referring to FIGS. 3A and 3B, depicted are graphical representations of shape features of waveforms (e.g., waveform morphology), according to non-limiting embodiments or aspects of methods for estimating ICP using NIRS. As shown, FIG. 3A illustrates peak-based shape features, including peak height (P1ₚₖ) (normalized to 1), peak width (P1_{w}), peak prominence (P1ₚ) (the vertical distance between the peak and its lowest contour line), and peak location (P1pos), for an ACPW produced from signals measured using NIRS for the blood attribute of ΔHbO. FIG. 3B illustrates other shape features, including x-coordinate of the center of mass (COMₓ), y-coordinate of the center of mass (COM_{y}), and area under the curve (AUC), for the same ACPW as FIG. 3A.

With further reference to FIGS. 3A and 3B, defining and extracting physiologically relevant features (e.g., shape features) from the processed ACPWs provides the basis for estimating ICP. MATLAB's findpeaks function may be used to obtain the peak height (P1ₚₖ, normalized to 1 and used as a measure for model noise), peak position (P1ₚₒₛ), peak prominence (P1ₚ), and peak width (P1_{w}) of the ACPWs. The x- and y-coordinates of the center of mass (centroid) of the waveform, as shown in FIG. 3B, defined as COMₓ and COM_{y}, were also extracted from individual ACPWs and used as features. These may be produced by turning each ACPW into a polygon and calculating its centroid using the “polyshape” and “centroid” functions in MATLAB. During analysis, it was hypothesized that COMₓ, which describes waveform skewness, is related to blood pressure and ICP. A similar reasoning motivated COM_{y}. The area under the curve (AUC) of the waveform was also incorporated as a feature, as was mean arterial pressure (MAP). Similar to ICP, MAP was calculated over each 120-pulse window. The feature engineering resulted in eight interpretable and observable features for each processed ΔHbO and ΔHbT ACPW. If a feature was undetected, its value was set to 0, but it was still used.

For analysis, each ACPW dataset was randomly sampled into five cross validation (CV) sets of 80% training and 20% testing. For each CV set, all animals were included. Random sampling ensured that learning became NHP and trial independent, while CV alleviated overfitting. Python's scikit-learn toolbox's random forest (RF) regression algorithm was used as the ICP estimator. The RF algorithm learns a set number of decision trees on a randomly sampled subset of the features using a randomly sampled subset of the training data with replacement (e.g., bootstrapping). A total of 100 trees, or estimators, were learned using this bootstrapping method. Every tree received 50% of the features and 80% of the dataset for training. All other hyperparameters were kept as default. These hyperparameters were chosen empirically to maximize performance while mitigating overfitting. The hyperparameter decision-making process used a random search to gauge approximate hyperparameter ranges, after which a per-parameter and joint-parameter optimization procedure followed. Random search hyperparameter tuning may also be used to optimize machine learning models. One significant parameter was the number of estimators, or trees. Each of the 100 trees splits its subset of data until all leaves are pure to maximum depth. Each tree receives four randomly sampled features for learning. Gini impurity was used as the measure of node split quality. For each CV split (fold), the RF algorithm was trained on the training split and tested on the testing split. During testing, estimated ICP values (ICPₑₛₜ) were compared with invasively measured ICP values (ICPᵢₙᵥ). ICPᵢₙᵥ values were used as ground truth labels. The performance of the model was quantified using the coefficient of determination (r²), mean squared error (MSE), and 95% confidence interval (CI).

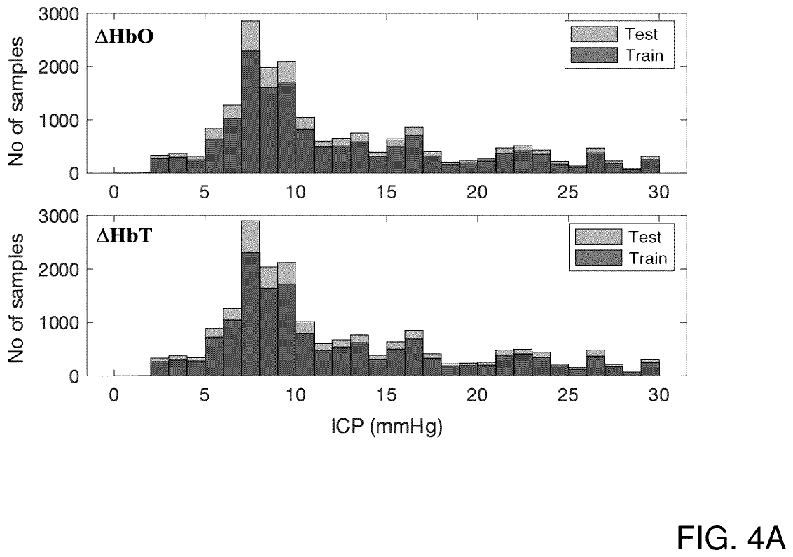

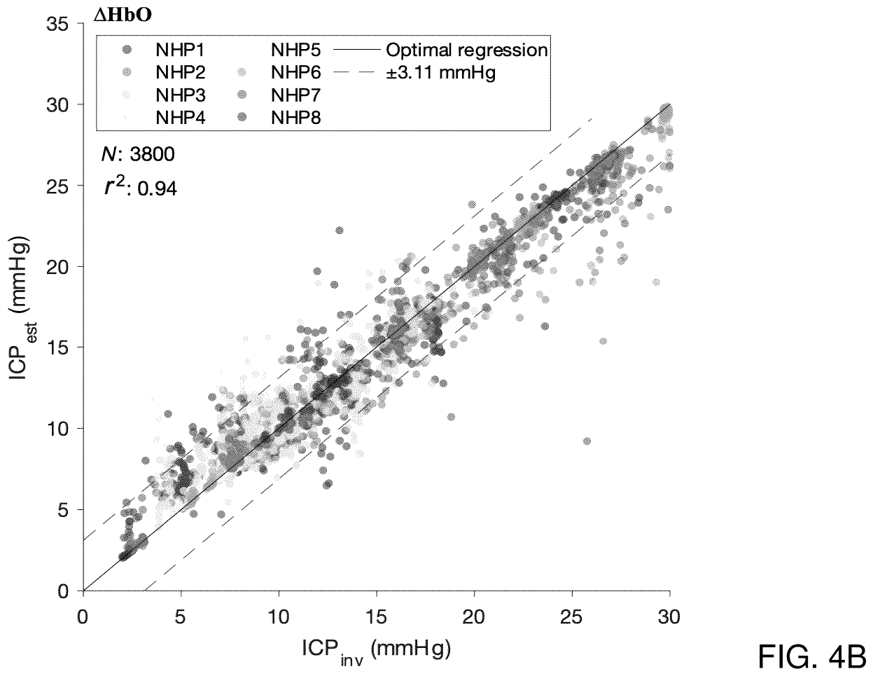

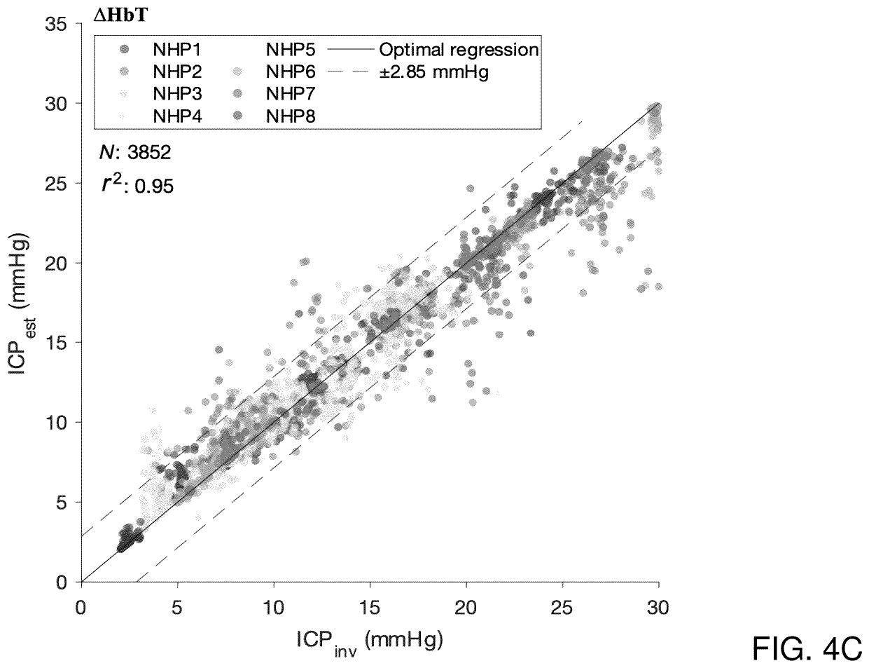

Referring to FIG. 4A, depicted are histograms of ICP distribution for training and test datasets for waveforms of light-based signals associated with blood attributes, according to some non-limiting embodiments or aspects of the present disclosure. In particular, FIG. 4A shows histograms of ICP distribution for ΔHbO and ΔHbT (produced from NIRS measurement). As shown, more data were available at lower ICP values, especially between 5 and 10 mmHg.

Referring to FIGS. 4B and 4C, depicted are correlation plots illustrating correlation between ICPₑₛₜ (determined from waveforms of light-based signals associated with blood attributes) and ICPᵢₙᵥ, according to some non-limiting embodiments or aspects of the present disclosure. In particular, FIG. 4B depicts a correlation plot illustrating correlation between ICPₑₛₜ determined from ΔHbO (related to a NIRS technique) and ICPᵢₙᵥ. FIG. 4C depicts a correlation plot illustrating correlation between ICPₑₛₜ determined from ΔHbT (related to a NIRS technique) and ICPᵢₙᵥ. Strong r² for all methods suggests that the model performs well on ICP estimation. Estimation performance drops for higher ICP values across all modalities (e.g., blood attributes) due to a lower availability of high ICP data for training. Within the 0 to 30 mmHg range of ICP values, available training and testing data were skewed toward lower ICP values, as shown in FIG. 4A.

With further reference to FIGS. 4B and 4C, to compare performance differences between the different types of light-based signals that are associated with blood attributes, the coefficient of determination (r²) and MSE were used as evaluation metrics, along with a 95% CI. A fivefold CV was performed individually on the ΔHbO and ΔHbT associated light-based signals. Analysis determined a mean fold of r²=0.937 (averaged over five folds) and r²=0.946 for the ACPWs of ΔHbO and ΔHbT, respectively, with a fivefold r² standard deviation of r²_{std}=0.003 (ΔHbO) and r²_{std}=0.004 (ΔHbT). Analysis determined a mean fold MSE=2.703 and 2.301 mmHg² with a fivefold standard deviation of MSE_{std}=0.133 and 0.163 mmHg² for ΔHbO and ΔHbT, respectively. Analysis indicated ICP can be estimated using within an MSE of <3 mmHg² when using NIRS-measured, hemoglobin-based waveforms.

All ICP extractions from non-invasive measurements of blood attribute-related signals show a good correlation between estimated and invasively measured ICP. Outliers were more common at higher ICP values for which less data were available for training, as shown in the histograms of FIG. 4A.

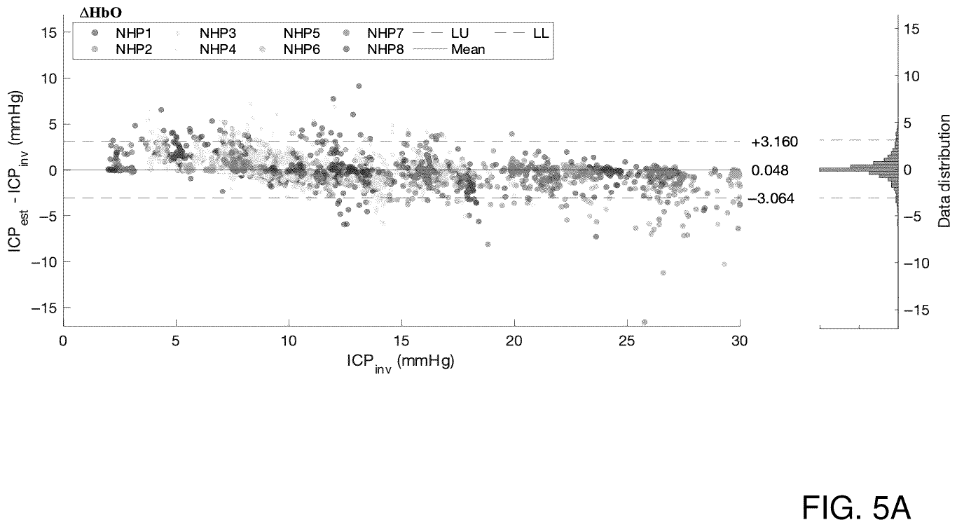

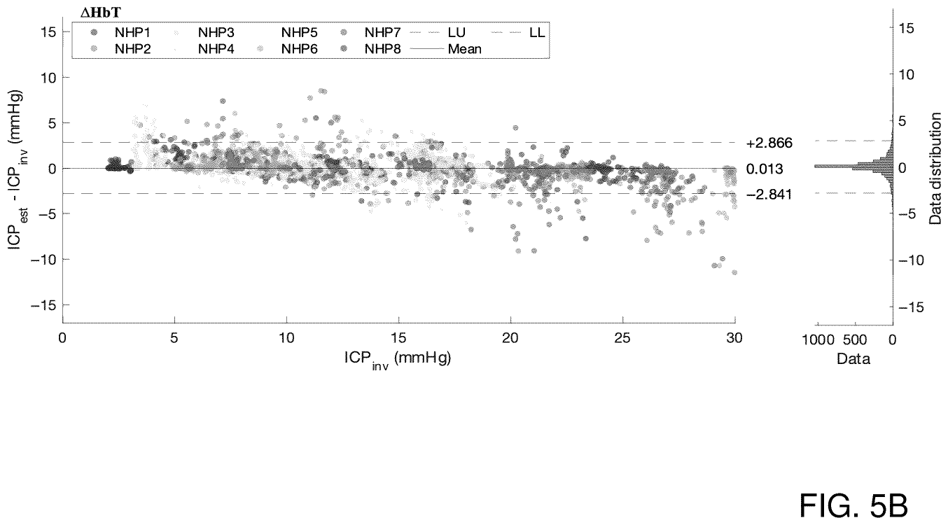

Referring to FIGS. 5A and 5B, depicted are a set of two Bland-Altman plots for ΔHbO and ΔHbT, according to some non-limiting embodiments or aspects of a method for estimating ICP using NIRS. In particular, FIG. 5A depicts a Bland-Altman plot for ΔHbO and FIG. 5B depicts a Bland-Altman plot for ΔHbT. The plots indicate the level of agreement between ICPₑₛₜ and ICPᵢₙᵥ and further confirm the clear fit of the trained model for estimating ICP in comparison to the invasively measured ICP. The plots show a 95% CI of agreement between ICPₑₛₜ and ICPᵢₙᵥ of [−3.064 3.160] mmHg with a mean of 0.048 mmHg for ΔHbO, and a 95% CI of [−2.841 2.866] mmHg with a mean of 0.013 mmHg for ΔHbT. As shown in the graphs, LU represents the upper limit of agreement and LL represents the lower limit of agreement, where a limit of agreement is defined by the mean difference ±1.96 SD (standard deviation) of differences.

With further reference to the analysis depicted in FIGS. 5A and 5B, Table 1 (shown below) summarizes the results (e.g., error metrics) across all modalities. When considering the performance of ΔHbO against ΔHbT, ΔHbT performs slightly better than ΔHbO, but both perform well in estimating ICP. ΔHbO and ΔHbT have a 0.009 difference in fivefold mean r² and a 0.402 mmHg² difference in fivefold mean MSE. The 95% CI metric across the modalities echoes this difference in model fit performance. NIRS performs comparatively well against DCS-based techniques, but is cheaper and easier to use.

TABLE 1

| Modality | r² | MSE | r² | MSE_{std} | 95% CI | Mean |
|---|---|---|---|---|---|---|
| ΔHbT | 0.946 | 2.301 | 0.004 | 0.163 | [−2.841 2.866] | 0.013 |
| ΔHbO | 0.937 | 2.703 | 0.003 | 0.133 | [−3.064 3.160] | 0.048 |

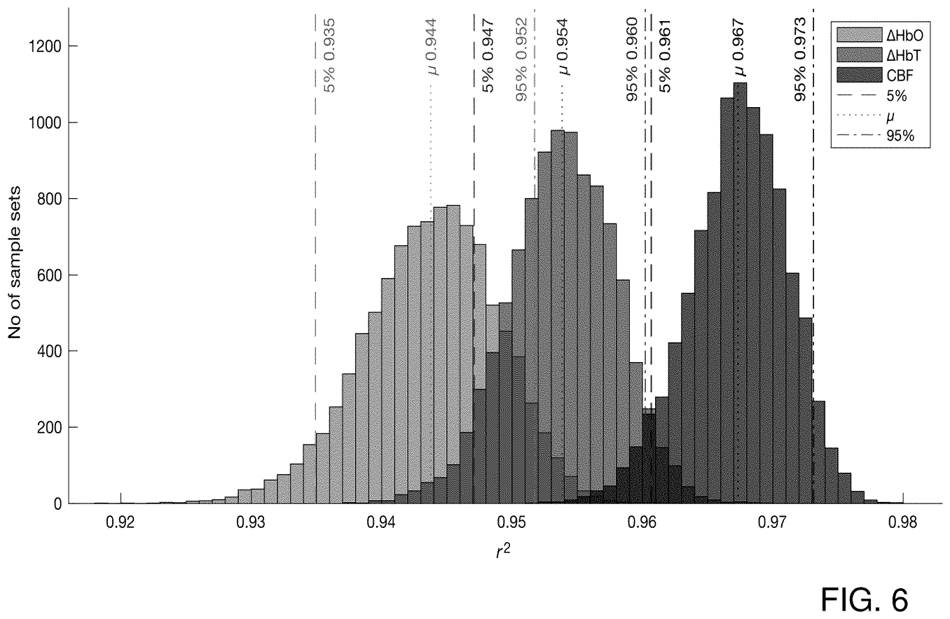

Referring to FIG. 6, depicted are histograms of bootstrapped r² scores for ΔHbO and ΔHbT (for NIRS-based techniques) and CBF (for DCS-based techniques) when used as modalities for training machine learning models to estimate ICP, according to some non-limiting embodiments or aspects of the present disclosure. As shown, bootstrapping used 50% of test samples drawn 10,000 times with replacement. The test data from the best performing CV split, per modality, are used. This results in sample mean r² slightly above the mean CV r² are reported. The score for r² is calculated for each set of samples across all modalities (ΔHbO, ΔHbT, and CBF). A significance level p≤0.05 is used. NIRS methods are comparable to DCS-based techniques in overall performance, but are easier and cheaper to use than DCS-based techniques.

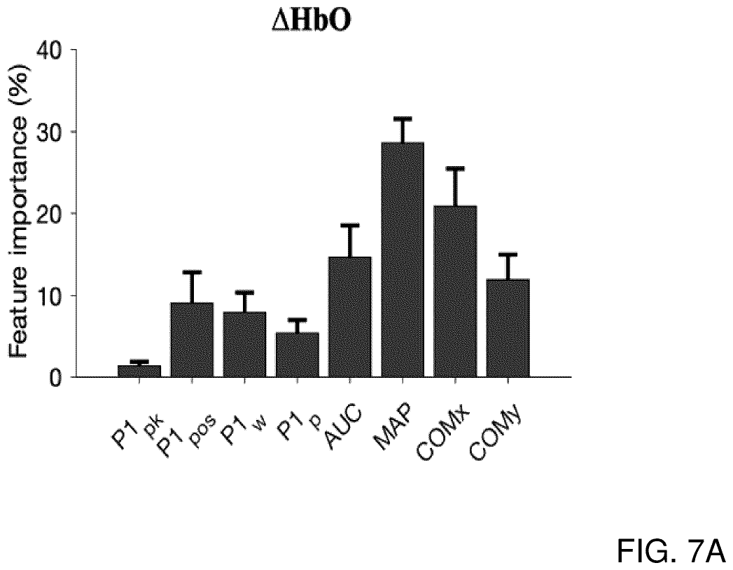

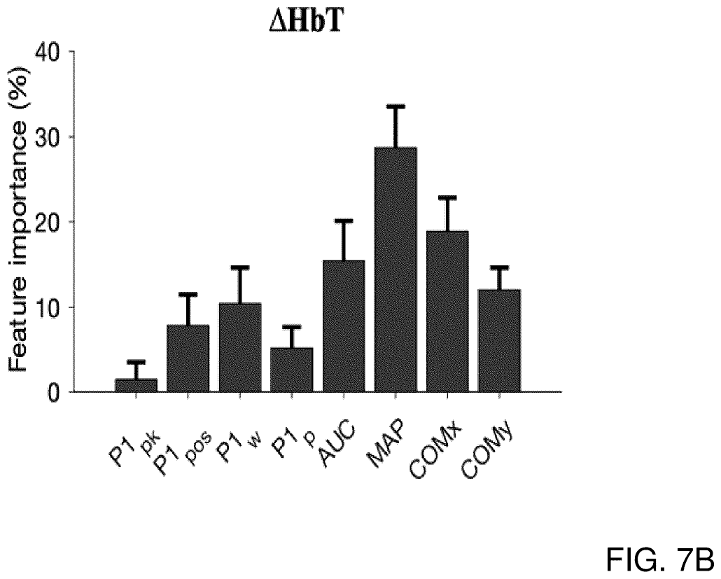

With further reference to FIG. 6, distributions of r² scores with 5%, mean (μ), and 95% thresholds are shown. For all modalities, the waveform shape feature relevance contributing to ICP extraction was evaluated. The relative magnitude of feature importance for ΔHbO and ΔHbT is shown in FIGS. 7A and 7B. Overall, MAP and COMₓ were the most relevant features used in the training of each model (e.g., estimator tree). AUC, COMₓ, and MAP accounted for ˜15%, ˜20%, and ˜28% of node splits per tree, respectively. Meanwhile, COM_{y} represented ˜11% of node splits, whereas waveform peak position represented ˜9%. The remaining three features represented <7% of node splits per tree. The standard deviation of features used between trees is shown by the error bars of each feature bar. AUC importance for ΔHbO and ΔHbT was approximately the same at around 15% to 18%. COMₓ and COM_{y} were less important, but still important, particularly more for ΔHbT compared with ΔHbO. MAP was the most important feature across modalities, with the level of importance being approximately the same. For ΔHbT, MAP represented ˜29% of splits per tree compared with ˜28% for ΔHbO. The importance of the height of the peak, which was normalized, is used as a proxy to measure the importance of an unrelated peak.

A more evenly balanced dataset was also tested for ΔHbO. Balancing was performed by randomly removing half of all ICP values between 5 and 10 mmHg. This balanced dataset had ˜23% less data than the skewed dataset. For ΔHbO, r² dropped by 0.6% from 0.937 to 0.932, MSE increased by 22.1% from 2.703 to 3.299 mmHg², and mean 95% CI increased by [12.3% 11.5%] from [−3.064 3.160] to [−3.441 3.523] mmHg with a 14.6% change in mean from 0.048 to 0.041 mmHg. When MAP was removed as a training feature, the importance of the four peak features was increased, and the importance of AUC and COMₓ was maintained. Performance may be improved with MAP as an added feature, but ICP can still be quantified to <˜5 mmHg for hemoglobin-based estimators (using NIRS techniques). The results indicate that NIRS is an effective alternative to DCS in terms of noninvasive ICP.

In building an NHP-based dataset of ICP-dependent ACPW features, it was observed that ΔHbO and ΔHbT cardiac pulse waveforms followed a canonical cardiac pulse arch shape. Using 120-pulse averaging and Kalman filtering improved SNR to the extent that meaningful feature extraction could be performed. The number of pulses averaged over was set to 120 to include enough pulses for appropriate waveform and ICP averaging. Averaging may assist in removing noise in the signal waveforms. Signal preprocessing and filtering may also assist with leveraging the raw signals extracted from the patient. Preprocessing and filtering may provide the feature extraction method with low-noise signals while not overfiltering or losing valuable information. Clean signals improve relevant feature-value-to-ICP mappings for the machine learning model to learn.

Tuning of the signal preprocessing and filtering parameters may be done empirically across the cardiac waveform averaging, waveform normalization, z-score rejection, and Kalman filtering methods. 120 consecutive pulses were found to be a suitable averaging amount to reduce signal to noise, whereas a 20-pulse averaging window shift struck a balance between obtaining ample amounts of data for training and testing while reducing data replication. Waveform normalization, z-score rejection, and Kalman filtering all worked to remove data outliers while maintaining a feature distribution that supported model generalization.

Referring to FIGS. 7A and 7B, depicted are histograms illustrating feature statistical importance (including for shape features of waveforms) in estimating ICP based on waveforms associated with the blood attributes of ΔHbO and ΔHbT, according to some non-limiting embodiments or aspects of the present disclosure. In particular, FIG. 7A depicts a histogram illustrating feature statistical importance for ΔHbO, and FIG. 7B depicts a histogram illustrating feature statistical importance for ΔHbT. As shown, MAP is strongest across all modalities. AUC and COMₓ are relevant for estimation, and peak-based features (P1ₚₒₛ, P1_{w}, and P1ₚ) may also be important. The analysis suggests that MAP on its own, though relevant, is not the only feature driving decision tree learning. This gives weight to other shape features, such as AUC and COMₓ. Extracting specific features from data may improve model generalizability for small sample sizes. The FDA's Good Machine Learning Practice for Medical Device Development: Guiding Principles highlights the importance of human interpretability of models and their outputs in clinical settings. As such, the features engineered herein (e.g., waveform shape features) are more interpretable when compared with machine-engineered features sometimes used in complex machine learning and neural network-based methods. The engineered features described herein also expressed themselves relatively predictably across modalities (e.g., blood attributes).

With further reference to the foregoing figures, random forest regressor models (RF) have a relatively low risk of overfitting with an increase in estimators due to their use of multiple weak and unpruned learners. Each tree in the RF ensemble does overfit to the data and the features that it receives, but the averaged result of the ensembled trees produces a regression prediction that is low in variance and bias. Ultimately, this means that the more trees that are used in the ensemble, the less likely the model is to overfit. The above-described approach is more computationally efficient and produces comparable results to other techniques.

Referring now to FIG. 8, FIG. 8 is a diagram of an example environment 800 in which devices, systems, and/or methods, described herein, may be implemented. As shown in FIG. 8, environment 800 may include modeling system 802, memory 804, computing device 806, and communication network 808. Modeling system 802, memory 804, and computing device 806 may interconnect (e.g., establish a connection to communicate) via wired connections, wireless connections, or a combination of wired and wireless connections.

Modeling system 802 may include one or more computing devices configured to communicate with memory 804 and/or computing device 806, at least partly over communication network 808. Modeling system 802 may be configured to receive data to train one or more machine learning models (e.g., random forest regressor models) and use one or more trained machine learning models to generate an output. Modeling system 802 may include, be included in a same system as, or be in communication with memory 804. Modeling system 802 may include a system for performing NIRS on a patient, or may be communicatively connected to a system for performing NIRS on a patient.

Memory 804 may include one or more computing devices configured to communicate with modeling system 802 and/or computing device 806 at least partly over communication network 808. Memory 804 may be configured to store data (in one or more non-transitory computer readable storage media) associated with shape features of waveforms, measured data of light-based signals using NIRS associated with one or more blood attributes, patient record data, and/or the like. Memory 804 may communicate with and/or be included in a same system as modeling system 802.

Computing device 806 may include one or more processors that are configured to communicate with modeling system 802 and/or memory 804 at least partly over communication network 808. Computing device 806 may be associated with a user and may include at least one user interface for transmitting data to and receiving data from modeling system 802 and/or memory 804. For example, computing device 806 may show, on a display of computing device 806, one or more outputs of trained machine learning models executed by modeling system 802. By way of further example, one or more inputs for trained machine learning models may be determined or received by modeling system 802 via a user interface of computing device 806. Computing device 806 may have an input component for a user to input data that may be used as an input for trained machine learning models.

Communication network 808 may include one or more wired and/or wireless networks over which the systems and devices of environment 800 may communicate. For example, communication network 808 may include a cellular network (e.g., a long-term evolution (LTE®) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

The number and arrangement of devices and networks shown in FIG. 8 are provided as an example. There may be additional devices and/or networks, fewer devices and/or networks, different devices and/or networks, or differently arranged devices and/or networks than those shown in FIG. 8. Furthermore, two or more devices shown in FIG. 8 may be implemented within a single device, or a single device shown in FIG. 8 may be implemented as multiple, distributed devices. Additionally or alternatively, a set of devices (e.g., one or more devices) of environment 800 may perform one or more functions described as being performed by another set of devices of environment 800.

In some non-limiting embodiments or aspects, modeling system 802 may include one or more processors that are programmed or configured to train machine learning models based on NIRS-generated waveform data. For example, modeling system 802 may generate first waveform data (e.g., data representative of one or more waveforms at a first point in time) using NIRS to measure at least one light-based signal (e.g., intensity) in each patient of a plurality of patients, wherein the first waveform data includes a plurality of waveforms, and wherein each waveform of the plurality of waveforms is associated with at least one blood attribute (e.g., change in oxygenated hemoglobin concentration (ΔHbO), change in deoxygenated hemoglobin concentration (ΔHb), change in total hemoglobin concentration (ΔHbT), etc.). The first waveform data generated by modeling system 802 may be used to train one or more machine learning models (e.g., random forest regressor models) to estimate ICP based on waveforms of the first waveform data. Modeling system 802 may train at least one machine learning model based on the first waveform data to produce at least one trained machine learning model. The at least one trained machine learning model may be configured to generate an output of ICP based on waveform data (e.g., one or more waveforms thereof) associated with the at least one blood attribute that is input to the at least one trained machine learning model.

In some non-limiting embodiments or aspects, modeling system 802 may include one or more processors that are programmed or configured to estimate ICP using machine learning models trained on NIRS-generated waveform data. For example, modeling system 802 may generate second waveform data (e.g., data representative of one or more waveforms at a second point in time after the first point in time) using NIRS to measure at least one light-based signal in a first patient (which may or may not be included in the cohort of the plurality of patients used for training), wherein the second waveform data includes at least one waveform associated with the at least one blood attribute. The at least one blood attribute measured by NIRS for the second waveform data includes one or more of the same blood attributes used to train the machine learning model (e.g., the at least one blood attribute measured by NIRS for the second waveform data may be the same as the at least one blood attribute measured by NIRS for the first waveform data). Modeling system 802 may determine an estimated ICP in the first patient based on the at least one trained machine learning model.

In some non-limiting embodiments or aspects, modeling system 802 may include one or more processors that are programmed or configured to determine an estimated ICP based on the second waveform data and the at least one trained machine learning model. For example, modeling system 802 may input at least a portion (e.g., data of one or more waveforms) of the second waveform data to the at least one trained machine learning model. Modeling system 802 may further generate an output from the at least one trained machine learning model including the estimated ICP based on at least one shape feature (e.g., area under the curve (AUC), x-coordinate of the center of mass (COMₓ), y-coordinate of the center of mass (COM_{y}), peak height, peak width, peak location, etc.) of the at least one waveform of the second waveform data. Modeling system 802 may generate the output from the at least one trained machine learning model based on a plurality of different shape features.

In some non-limiting embodiments or aspects, modeling system 802 may pre-process and aggregate the first waveform data used to train the at least one machine learning model. For example, modeling system 802 may remove data outliers from the first waveform data using a preprocessing technique (e.g., normalization, z-score rejection, Kalman filtering, etc.). Modeling system 802 may further, when generating the first waveform data, generate a subset of the plurality of waveforms for each patient of the plurality of patients using NIRS to measure a plurality of consecutive cardiac pulses, and determine an average cardiac waveform (ACPW) for said each patient based on the subset of the plurality of waveforms. The number of consecutive cardiac pulses may be more than fifty (e.g., 60 to 120) for determination of the ACPW, but it will be appreciated that two or more waveforms may be averaged to determine an ACPW.

In some non-limiting embodiments or aspects, modeling system 802 may include one or more processors programmed or configured to execute one or more processes based on the estimated ICP generated from the at least one trained machine learning model. For example, modeling system 802 may compare the estimated ICP to at least one predetermined threshold ICP (e.g., 20 mmHg). In response to the estimated ICP satisfying the at least one predetermined threshold ICP, modeling system 802 may generate at least one alert (e.g., visual, aural, or other sensory notification) to a computing device (e.g., a mobile device, a surgical monitor, etc.) associated with a healthcare personnel (e.g., a nurse, a surgical team, a doctor, an attendant, etc.) providing care (e.g., monitoring status, performing surgery, administering medicine, etc.) to the first patient. The alert may be configured to cause the computing device to perform one or more treatment processes in response to receiving the alert. Modeling system 802 may also perform at least one treatment (e.g., administering medication, controlling an intravenous solution rate, etc.) for the first patient based on the estimated ICP (e.g., to increase ICP if the estimated ICP is low, to decrease ICP if the estimated ICP is high, etc.).

In some non-limiting embodiments or aspects, modeling system 802 may determine mean arterial pressure (MAP) data of the first patient. When determining the estimated ICP in the first patient, modeling system 802 may input the MAP data to the at least one trained machine learning model and generate the output from the at least one trained machine learning model including the estimated ICP based on the MAP data and the at least one shape feature of the at least one waveform of the second waveform data.

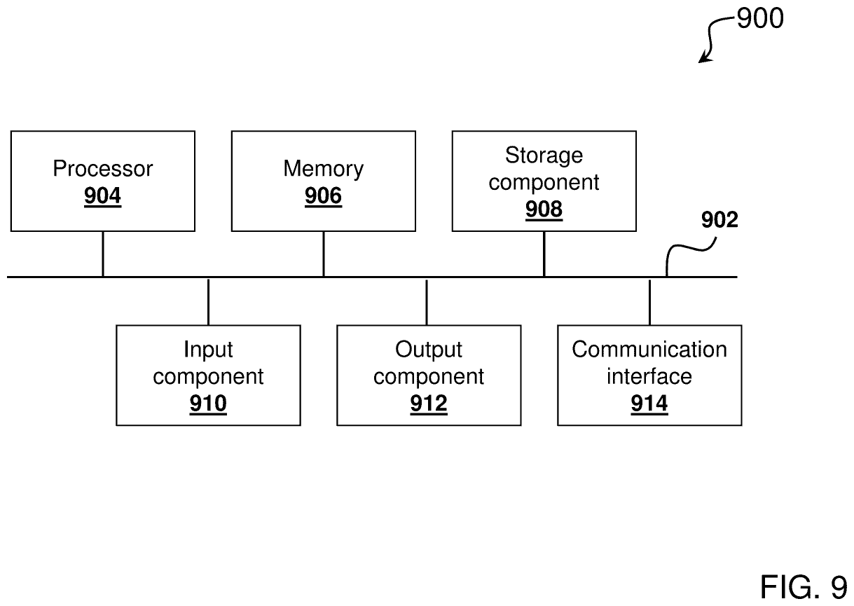

Referring now to FIG. 9, FIG. 9 is a diagram of example components of a device 900, according to some non-limiting embodiments or aspects. Device 900 may correspond to one or more devices of modeling system 802, memory 804, computing device 806, and/or communication network 808, as shown in FIG. 8. In some non-limiting embodiments or aspects, such systems or devices may include at least one device 900 and/or at least one component of device 900.

As shown in FIG. 9, device 900 may include bus 902, processor 904, memory 906, storage component 908, input component 910, output component 912, and communication interface 914. Bus 902 may include a component that permits communication among the components of device 900. In some non-limiting embodiments or aspects, processor 904 may be implemented in hardware, firmware, or a combination of hardware and software. For example, processor 904 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 906 may include random access memory (RAM), read only memory (ROM), and/or another type of dynamic or static storage device (e.g., flash memory, magnetic memory, optical memory, etc.) that stores information and/or instructions for use by processor 904.

Storage component 908 may store information and/or software related to the operation and use of device 900. For example, storage component 908 may include a hard disk (e.g., a magnetic disk, an optical disk, a magneto-optic disk, a solid state disk, etc.) and/or another type of computer-readable medium.

Input component 910 may include a component that permits device 900 to receive information, such as via user input (e.g., a touch screen display, a keyboard, a keypad, a mouse, a button, a switch, a microphone, etc.). Additionally, or alternatively, input component 910 may include a sensor for sensing information (e.g., a global positioning system (GPS) component, an accelerometer, a gyroscope, an actuator, etc.). Output component 912 may include a component that provides output information from device 900 (e.g., a display, a speaker, one or more light-emitting diodes (LEDs), etc.).

Communication interface 914 may include a transceiver-like component (e.g., a transceiver, a separate receiver and transmitter, etc.) that enables device 900 to communicate with other devices, such as via a wired connection, a wireless connection, or a combination of wired and wireless connections. Communication interface 914 may permit device 900 to receive information from another device and/or provide information to another device. For example, communication interface 914 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 900 may perform one or more processes described herein. Device 900 may perform these processes based on processor 904 executing software instructions stored by a computer-readable medium, such as memory 906 and/or storage component 908. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 906 and/or storage component 908 from another computer-readable medium or from another device via communication interface 914. When executed, software instructions stored in memory 906 and/or storage component 908 may cause processor 904 to perform one or more processes described herein. Additionally, or alternatively, hardwired circuitry may be used in place of or in combination with software instructions to perform one or more processes described herein. Thus, embodiments or aspects described herein are not limited to any specific combination of hardware circuitry and software.

The number and arrangement of components shown in FIG. 9 are provided as an example. In some non-limiting embodiments, device 900 may include additional components, fewer components, different components, or differently arranged components than those shown in FIG. 9. Additionally or alternatively, a set of components (e.g., one or more components) of device 900 may perform one or more functions described as being performed by another set of components of device 900.

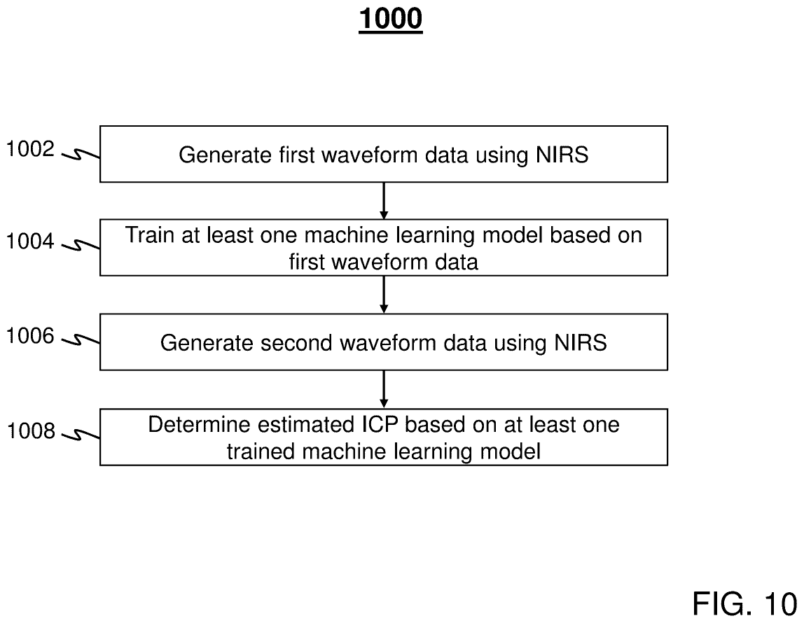

Referring now to FIG. 10, FIG. 10 is a flowchart of a non-limiting embodiment or aspect of a process 1000 for estimating ICP using NIRS, according to some non-limiting embodiments or aspects. The steps shown in FIG. 10 are for example purposes only. It will be appreciated that additional, fewer, different, and/or a different order of steps may be used in non-limiting embodiments or aspects. In some non-limiting embodiments or aspects, one or more of the steps of process 1000 may be performed (e.g., completely, partially, and/or the like) by modeling system 802. In some non-limiting embodiments or aspects, one or more of the steps of process 1000 may be performed (e.g., completely, partially, and/or the like) by another system, another device, another group of systems, or another group of devices, separate from or including modeling system 802.

As shown in FIG. 10, at step 1002, process 1000 may include generating first waveform data using NIRS. For example, modeling system 802 may generate first waveform data using NIRS to measure at least one light-based signal in each patient of a plurality of patients, wherein the first waveform data includes a plurality of waveforms, and wherein each waveform of the plurality of waveforms is associated with at least one blood attribute. The at least one blood attribute may include one or more of the following: change in oxygenated hemoglobin concentration (ΔHbO), change in deoxygenated hemoglobin concentration (ΔHb), change in total hemoglobin concentration (ΔHbT), and/or the like.

As shown in FIG. 10, at step 1004, process 1000 may include training at least one machine learning model based on the first waveform data. For example, modeling system 802 may train at least one machine learning model based on the first waveform data to produce at least one trained machine learning model. In some non-limiting embodiments or aspects, the at least one machine learning model (and, therefore, the at least one trained machine learning model) may include a random forest regressor model. The at least one trained machine learning model may be configured to generate an output of ICP based on one or more waveforms associated with the at least one blood attribute that are input to the at least one trained machine learning model.

In some non-limiting embodiments or aspects, step 1004 may include additional filtering and pre-processing of the first waveform data. For example, modeling system 802 may remove data outliers from the first waveform data using a preprocessing technique including, but not limited to, at least one of the following: normalization, z-score rejection, Kalman filtering, or any combination thereof. By way of further example, modeling system 802 may generate a subset of the plurality of waveforms for each patient of the plurality of patients using NIRs to measure a plurality of consecutive cardiac pulses, and may determine an average cardiac waveform (ACPW) for said each patient based on the subset of the plurality of waveforms. The ACPW may be used as the representative waveform for each patient in the first waveform data. The number of consecutive cardiac pulses may be equal to or greater than fifty (e.g., 60 to 120) consecutive cardiac pulses.

As shown in FIG. 10, at step 1006, process 1000 may include generating second waveform data using NIRS. For example, modeling system 802 may generate second waveform data using NIRS to measure at least one light-based signal in a first patient, wherein the second waveform data includes at least one waveform associated with the at least one blood attribute. In some non-limiting embodiments or aspects, the first patient may be a subject for which an estimated ICP is generated, which may be further used to generate alerts, initiate a treatment, store ICP data, and/or the like.

As shown in FIG. 10, at step 1008, process 1000 may include determining an estimated ICP based on at least one trained machine learning model. For example, modeling system 802 may determine an estimated ICP in the first patient based on the at least one trained machine learning model. While determining the estimated ICP in the first patient based on the at least one trained machine learning model, modeling system 802 may input at least a portion of the second waveform data to the at least one trained machine learning model, and generate an output from the at least one trained machine learning model including the estimated ICP based on at least one shape feature of the at least one waveform of the second waveform data. The at least one shape feature of the at least one waveform of the second waveform data may include, but is not limited to, area under the curve (AUC), x-coordinate of the center of mass (COMₓ), y-coordinate of the center of mass (COM_{y}), peak height (e.g., amplitude at peak), peak width (e.g., width of curve at x-value of peak), peak location (e.g., x- and/or y-coordinate of the peak), and/or the like.

In some non-limiting embodiments or aspects, step 1008 may further include determining an estimated ICP at least partly based on mean arterial pressure (MAP) data. For example, modeling system 802 may determine MAP data of the first patient, input the MAP data to the at least one trained machine learning model, and generate the output from the at least one trained machine learning model including the estimated ICP based on the MAP data and the at least one shape feature of the at least one waveform of the second waveform data.

In some non-limiting embodiments or aspects, step 1008 may further include executing one or more process based on the estimated ICP. For example, modeling system 802 may compare the estimated ICP to at least one predetermined threshold ICP and, in response to the estimated ICP satisfying the at least one predetermined threshold ICP, generate at least one alert to a computing device associated with a healthcare personnel providing care to the first patient. By way of further example, modeling system 802 may perform at least one treatment for the first patient based on the estimated ICP.

Although non-limiting embodiments have been described in detail for the purpose of illustration based on what may be considered to be the most practical and preferred embodiments, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A computer-implemented method comprising:
generating, with at least one processor, first waveform data using near-infrared spectroscopy (NIRS) to measure at least one light-based signal in each patient of a plurality of patients, wherein the first waveform data comprises a plurality of waveforms, and wherein each waveform of the plurality of waveforms is associated with at least one blood attribute;
training, with at least one processor, at least one machine learning model based on the first waveform data to produce at least one trained machine learning model, wherein the at least one trained machine learning model is configured to generate an output of intracranial pressure (ICP) based on one or more waveforms associated with the at least one blood attribute that is input to the at least one trained machine learning model;
generating, with at least one processor, second waveform data using NIRS to measure at least one light-based signal in a first patient, wherein the second waveform data comprises at least one waveform associated with the at least one blood attribute; and
determining, with at least one processor, an estimated ICP in the first patient based on the at least one trained machine learning model, wherein determining the estimated ICP in the first patient based on the at least one trained machine learning model comprises:
inputting at least a portion of the second waveform data to the at least one trained machine learning model; and
generating an output from the at least one trained machine learning model comprising the estimated ICP based on at least one shape feature of the at least one waveform of the second waveform data.

2. The method of claim 1, wherein generating the first waveform data further comprises:
removing, with at least one processor, data outliers from the first waveform data using a preprocessing technique comprising at least one of the following: normalization, z-score rejection, Kalman filtering, or any combination thereof.

3. The method of claim 1, further comprising:
comparing, with at least one processor, the estimated ICP to at least one predetermined threshold ICP; and
in response to the estimated ICP satisfying the at least one predetermined threshold ICP, generating, with at least one processor, at least one alert to a computing device associated with a healthcare personnel providing care to the first patient.

4. The method of claim 1, further comprising performing, with at least one processor, at least one treatment for the first patient based on the estimated ICP.

5. The method of claim 1, wherein the at least one machine learning model comprises a random forest model.

6. The method of claim 1, further comprising determining, with at least one processor, mean arterial pressure (MAP) data of the first patient, wherein determining the estimated ICP in the first patient based on the at least one trained machine learning model further comprises:
inputting the MAP data to the at least one trained machine learning model; and
generating the output from the at least one trained machine learning model comprising the estimated ICP based on the MAP data and the at least one shape feature of the at least one waveform of the second waveform data.

7. The method of claim 1, wherein the at least one shape feature of the at least one waveform comprises at least one of the following: area under the curve (AUC), x-coordinate of the center of mass (COMₓ), y-coordinate of the center of mass (COM_{y}), peak height, peak width, peak location, or any combination thereof.

8. The method of claim 7, wherein generating the output comprising the estimated ICP further comprises:
generating the output from the at least one trained machine learning model comprising the estimated ICP based on the at least one shape feature of the at least one waveform of the second waveform data, wherein the at least one shape feature comprises a plurality of different shape features.

9. The method of claim 1, wherein generating the first waveform data further comprises:
generating, with at least one processor, a subset of the plurality of waveforms for each patient of the plurality of patients using NIRS to measure a plurality of consecutive cardiac pulses; and
determining, with at least one processor, an average cardiac waveform (ACPW) for said each patient based on the subset of the plurality of waveforms.

10. The method of claim 9, wherein the plurality of consecutive cardiac pulses numbers in a range of 60 to 120 consecutive cardiac pulses.

11. The method of claim 1, wherein the at least one blood attribute comprises at least one of the following: change in oxygenated hemoglobin concentration (ΔHbO), change in total hemoglobin concentration (ΔHbT), or any combination thereof.

12. A system comprising at least one processor programmed or configured to:
generate first waveform data using near-infrared spectroscopy (NIRS) to measure at least one light-based signal in each patient of a plurality of patients, wherein the first waveform data comprises a plurality of waveforms, and wherein each waveform of the plurality of waveforms is associated with at least one blood attribute;
train at least one machine learning model based on the first waveform data to produce at least one trained machine learning model, wherein the at least one trained machine learning model is configured to generate an output of intracranial pressure (ICP) based on one or more waveforms associated with the at least one blood attribute that is input to the at least one trained machine learning model;
generate second waveform data using NIRS to measure at least one light-based signal in a first patient, wherein the second waveform data comprises at least one waveform associated with the at least one blood attribute; and
determine an estimated ICP in the first patient based on the at least one trained machine learning model, wherein, while determining the estimated ICP in the first patient based on the at least one trained machine learning model, the at least one processor is further programmed or configured to:
input at least a portion of the second waveform data to the at least one trained machine learning model; and
generate an output from the at least one trained machine learning model comprising the estimated ICP based on at least one shape feature of the at least one waveform of the second waveform data.

13. The system of claim 12, wherein the at least one shape feature of the at least one waveform comprises at least one of the following: area under the curve (AUC), x-coordinate of the center of mass (COMₓ), y-coordinate of the center of mass (COM_{y}), peak height, peak width, peak location, or any combination thereof.

14. The system of claim 13, wherein, while generating the output comprising the estimated ICP, the at least one processor is programmed or configured to:
generate the output from the at least one trained machine learning model comprising the estimated ICP based on the at least one shape feature of the at least one waveform of the second waveform data, wherein the at least one shape feature comprises a plurality of different shape features.

15. The system of claim 12, wherein the at least one blood attribute comprises at least one of the following: change in oxygenated hemoglobin concentration (ΔHbO), change in total hemoglobin concentration (ΔHbT), or any combination thereof.

16. A computer program product comprising at least one non-transitory computer-readable medium comprising one or more instructions that, when executed by at least one processor, cause the at least one processor to:
generate first waveform data using near-infrared spectroscopy (NIRS) to measure at least one light-based signal in each patient of a plurality of patients, wherein the first waveform data comprises a plurality of waveforms, and wherein each waveform of the plurality of waveforms is associated with at least one blood attribute;
train at least one machine learning model based on the first waveform data to produce at least one trained machine learning model, wherein the at least one trained machine learning model is configured to generate an output of intracranial pressure (ICP) based on one or more waveforms associated with the at least one blood attribute that is input to the at least one trained machine learning model;
generate second waveform data using NIRS to measure at least one light-based signal in a first patient, wherein the second waveform data comprises at least one waveform associated with the at least one blood attribute; and
determine an estimated ICP in the first patient based on the at least one trained machine learning model, wherein the one or more instructions that cause the at least one processor to determine the estimated ICP in the first patient based on the at least one trained machine learning model cause the at least one processor to:
input at least a portion of the second waveform data to the at least one trained machine learning model; and
generate an output from the at least one trained machine learning model comprising the estimated ICP based on at least one shape feature of the at least one waveform of the second waveform data.

17. The computer program product of claim 16, wherein the one or more instructions further cause the at least one processor to:
compare the estimated ICP to at least one predetermined threshold ICP; and
in response to the estimated ICP satisfying the at least one predetermined threshold ICP, generate at least one alert to a computing device associated with a healthcare personnel providing care to the first patient.

18. The computer program product of claim 16, wherein the one or more instructions further cause the at least one processor to determine mean arterial pressure (MAP) data of the first patient, and wherein the one or more instructions that cause the at least one processor to determine the estimated ICP in the first patient based on the at least one trained machine learning model cause the at least one processor to:
input the MAP data to the at least one trained machine learning model; and
generate the output from the at least one trained machine learning model comprising the estimated ICP based on the MAP data and the at least one shape feature of the at least one waveform of the second waveform data.

19. The computer program product of claim 16, wherein the at least one shape feature of the at least one waveform comprises at least one of the following: area under the curve (AUC), x-coordinate of the center of mass (COMₓ), y-coordinate of the center of mass (COM_{y}), peak height, peak width, peak location, or any combination thereof.

20. The computer program product of claim 16, wherein the at least one blood attribute comprises at least one of the following: change in oxygenated hemoglobin concentration (ΔHbO), change in total hemoglobin concentration (ΔHbT), or any combination thereof.
